(19) United States
(12) Patent Application Publication
(10) Pub. No.: US 2011/0207793 A1
(43) Pub. Date: Aug. 25, 2011

(54) 1-AMINOCYCLOHEXANE DERIVATIVES FOR THE TREATMENT OF SLEEP DISORDERS.

(76) Inventors: Barbara Ellers-Lenz, Morfelden-Walldorf (DE); Tanja Rosenberg, Hamburg (DE); Hagen Kruger, Hamburg (DE); Michael Althaus, Schoneck (DE)

(21) Appl. No.: 12/737,092

(22) PCT Filed: Jun. 10, 2009
(86) PCT No.: PCT/EP2009/004184

§ 371 Date: Feb. 22, 2011

(30) Foreign Application Priority Data
Jun. 12, 2008 (EP) 08010749.3
Dec. 6, 2008 (US) 61131789

(51) Int. Cl. A61K 31/13; A61K 31/4045; A61P 25/00

(52) U.S. Cl. 514/415; 514/579

(57) ABSTRACT

The present invention relates to the treatment of an individual afflicted with sleep disorders associated with tinnitus and/or neurological diseases comprising administering to the individual an effective amount of a 1-amino-alkylcyclohexane derivative.

### FIELD OF THE INVENTION

The present invention relates to the treatment of an individual afflicted with sleep disorders per se, as well as sleep disorders associated with tinnitus and/or neurological diseases, comprising administering to the individual an effective amount of a 1-amino-alkylcyclohexane derivative.

### BACKGROUND OF THE INVENTION

This invention relates to methods of treating patients afflicted with sleep disorders per se, as well as sleep disorders associated with tinnitus and/or neurological diseases.

Tinnitus is commonly referred to as ‘ringing in the ears’—the perception of sounds in the absence of an external source of acoustic signals. Tinnitus has been defined as “the perception of a sound which results exclusively from the activity within the nervous system without any corresponding mechanical, vibratory activity within the cochlea, that is, tinnitus as an auditory phantom perception” (Jastreboff et al., J Am Acad Audiol 2000; 11(3): 162-177).

The pathophysiology of subjective tinnitus is poorly understood and a definitive pathogenesis of tinnitus is unknown. Many environmental and substance-induced factors may cause tinnitus. Among the most frequently cited factors are acute acoustic trauma, occupational noise, and recreational music. In general, tinnitus seems to be the result of neuronal dysfunction within the auditory pathway. This dysfunction is misleadingly perceived as sound by higher auditory centers and can lead to functional alterations within the auditory nervous system. Maladaptive functional changes in cortical structures could result in an altered balance between excitatory and inhibitory neurotransmission and may lead to more severe tinnitus. In all cases, a potential malfunction in auditory pathways and auditory cortex is related to the activity of the prefrontal cortex and limbic system.

In most cases (95%), the perceived tinnitus is purely subjective in nature, e.g. no physical source of acoustic signals can be identified and, therefore, cannot be heard externally. A physical examination is performed to exclude objective tinnitus, e.g. the patient's perception of sound is caused by a real source of sound waves, e.g. the sound from turbulent flow in blood vessels reaching the cochlea. Tinnitus may be classified according to duration of tinnitus and the degree of tinnitus expression (e.g. severity or annoyance of the tinnitus) (McCombe et al., Clin Otolaryngol 2001; 26(5): 388-393 and Davis et al., Epidemiology of Tinnitus. In: Tyler R, editor. Tinnitus Handbook. San Diego: Singular Publishing Group; 2000. p. 1-23). Regarding the impact of tinnitus, tinnitus may be severely annoying to the patient and may be accompanied by social and psychological complications.

Patients with tinnitus commonly complain of sleep disturbances with a published prevalence in a range up to 50% to 77% (Crönlein et al., Prog Brain Res 2007; 166: 227-33). Various aspects of sleep have been reported to be affected in tinnitus patients, including latency to sleep onset, sleep duration, duration of deep-sleep phases, sleep restfulness, or sleep efficiency (Burgos et al., Somnologie 2005;9 (3): 133-138). In severe cases, daytime wakefulness may also be impaired. Tinnitus patients with concurrent sleep disturbances often report a greater severity of their tinnitus, and many tinnitus patients conversely report a reduced tinnitus severity if their sleep is improved (Folmer/Griest, Am J Otolaryngol 2000; 21(5): 287-93). In accordance, tinnitus patients rated sleep most frequently when asked for conditions that reduce the severity of their tinnitus (Stouffer et al., Am J Otol, 1991; 12(3):188-94). Sleep of tinnitus patients may also be affected due to non-restorative sleep, an impairment of the sleep period described as non-refreshing sleep which is not typically associated with difficulties in initiating sleep or difficulties maintaining sleep.

For some patients, devices such as sound generators or masking music may provide a certain relief. Various drugs, including OTC-drugs, are also used to improve sleep in tinnitus patients. There are a number of disadvantages associated with such drugs when they are chronically used for the treatment of insomnia, and no therapy has been approved for the treatment of both tinnitus and sleep disturbances in patients with tinnitus. Thus, a need exists for pharmaceutical products which are effective in treating sleep disorders associated with tinnitus.

1-Amino-alkylcyclohexanes such as neramexane (also known as 1-amino-1,3,3,5,5-pentamethylcyclohexane) have been found to be useful in the therapy of various diseases especially in certain neurological diseases, including Alzheimer's disease and neuropathic pain. 1-Amino-alkylcyclohexanes such as neramexane are disclosed in detail in U.S. Pat. Nos. 6,034,134 and 6,071,966, the subject matter of which patents is hereby incorporated by reference. It is believed that the therapeutic action of 1-amino-alkylcyclohexanes such as neramexane is related to the inhibition of the effects of excessive glutamate at the N-methyl-D-aspartate (NMDA) receptors of nerve cells, for which reason the compounds are also categorized as NMDA antagonists, or NMDA receptor antagonists. Neramexane has also been disclosed to exhibit activity as an alpha9/alpha10 nicotinic receptor antagonist (Plazas, et al., Eur J Pharmacol., 2007 Jul. 2; 566(1-3):11-19).

U.S. Pat. No. 6,034,134 discloses that 1-amino-alkylcyclohexanes may be useful in the treatment of tinnitus due to their activity as NMDA receptor antagonists.

The instant inventors have discovered that 1-amino-alkylcyclohexanes, such as neramexane, are effective in treating sleep disorders associated with tinnitus.

Moreover, 1-amino-alkylcyclohexanes, such as neramexane, may also be effective in treating sleep disorders per se, as well as sleep disorders associated with neurological diseases such as neuropathic and chronic pain, fibromyalgia, or chronic fatigue syndrome.

### SUMMARY OF THE INVENTION

The present invention relates to a method for treating or preventing sleep disorders per se, as well as sleep disorders associated with tinnitus and/or neurological diseases in a subject in need thereof, comprising administering an effective amount of a 1-amino-alkylcyclohexane derivative (e.g., neramexane or a pharmaceutically acceptable salt thereof such as neramexane mesylate).

A further aspect of the invention relates to a method for treating or preventing sleep disorders associated with tinnitus and/or neurological diseases in a subject in need thereof, comprising administering an effective amount of a 1-amino-alkylcyclohexane derivative (e.g., neramexane or a pharmaceutically acceptable salt thereof such as neramexane mesylate), wherein the 1-amino-alkylcyclohexane derivative (e.g., neramexane or a pharmaceutically acceptable salt thereof such as neramexane mesylate) is administered in a range from about 5 mg to about 150 mg per day, including from about 5 mg to about 100 mg per day and from about 5 mg to about 75 mg per day or wherein the 1-amino-alkylcyclohexane derivative (e.g., neramexane or a pharmaceutically acceptable salt thereof such as neramexane mesylate) is administered at about 50 mg per day or about 75 mg per day.

A further aspect of the invention relates to a method for treating or preventing sleep disorders associated with tinnitus and/or neurological diseases in a subject in need thereof, comprising administering a 1-amino-alkylcyclohexane derivative (e.g., neramexane or a pharmaceutically acceptable salt thereof such as neramexane mesylate), wherein the 1-amino-alkylcyclohexane derivative (e.g., neramexane or a pharmaceutically acceptable salt thereof such as neramexane mesylate) is administered in a titration scheme which provides quick and safe attainment of an effective dose.

A further aspect of the invention relates to such a titration scheme wherein the 1-amino-alkylcyclohexane derivative (e.g., neramexane or a pharmaceutically acceptable salt thereof such as neramexane mesylate) is administered according to the following schedule: once daily at a dose of 25 mg per day for the first week, once daily at a dose of 50 mg per day for the second week, and, optionally, once daily at a dose of 75 mg per day for the third week.

A further aspect of the invention relates to such a titration scheme comprising up-titration of neramexane, or a pharmaceutically acceptable salt thereof, over a period of four weeks to achieve an effective dose of 50 mg per day.

A further aspect of the invention relates to such a titration scheme wherein neramexane or a pharmaceutically acceptable salt thereof is administered according to the following schedule: once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week.

A further aspect of the invention relates to such a titration scheme comprising up-titration of neramexane, or a pharmaceutically acceptable salt thereof, over a period of five weeks to achieve an effective dose of 75 mg per day.

A further aspect of the invention relates to such a titration scheme wherein neramexane or a pharmaceutically acceptable salt thereof is administered according to the following schedule: once daily at a dose of 12.5 mg per day for the first week, twice daily, wherein each dose is 12.5 mg for the second week, twice daily, wherein one dose is 12.5 mg and the other dose is 25 mg for the third week, and twice daily, wherein each dose is 25 mg for the fourth week, and twice daily, wherein each dose is 37.5 mg for the fifth week.

A further aspect of the invention relates to a 1-amino-alkylcyclohexane derivative (e.g., neramexane or a pharmaceutically acceptable salt thereof such as neramexane mesylate) for the treatment of an individual afflicted with sleep disorders associated with tinnitus and/or neurological diseases.

A further aspect of the invention relates to the use of a 1-amino-alkylcyclohexane derivative (e.g., neramexane or a pharmaceutically acceptable salt thereof such as neramexane mesylate) for the manufacture of a medicament for treatment or prevention of sleep disorders associated with tinnitus and/or neurological diseases.

A further aspect of the invention relates to a pharmaceutical composition for the treatment or prevention of sleep disorders associated with tinnitus and/or neurological diseases comprising a therapeutically effective amount of a 1-amino-alkylcyclohexane derivative (e.g., neramexane or a pharmaceutically acceptable salt thereof such as neramexane mesylate), and, optionally, at least one pharmaceutically acceptable carrier or excipient.

A further aspect of the invention relates to a pharmaceutical composition for the treatment or prevention of sleep disorders associated with tinnitus and/or neurological diseases comprising a therapeutically effective amount of a 1-amino-alkylcyclohexane derivative (e.g., neramexane or a pharmaceutically acceptable salt thereof such as neramexane mesylate) in an immediate or modified release formulation.

A further aspect of the invention relates to a method for treating or preventing sleep disorders associated with tinnitus and/or neurological diseases in a subject in need thereof, comprising administering a 1-amino-alkylcyclohexane derivative (e.g., neramexane or a pharmaceutically acceptable salt thereof such as neramexane mesylate) and at least one additional pharmaceutical agent which has been shown to be effective in treating or preventing sleep disorders.

A further aspect of the invention relates to a method for treating or preventing sleep disorders associated with tinnitus and/or neurological diseases in a subject in need thereof, comprising administering to the individual a 1-amino-alkylcyclohexane derivative (e.g., neramexane or a pharmaceutically acceptable salt thereof such as neramexane mesylate) and at least one additional pharmaceutical agent selected from melatonin, and melatonin receptor agonists.

A further aspect of the invention relates to a pharmaceutical composition comprising a therapeutically effective amount of a 1-amino-alkylcyclohexane derivative (e.g., neramexane or a pharmaceutically acceptable salt thereof such as neramexane mesylate) in combination with an additional pharmaceutical agent which has been shown to be effective for the treatment or the prevention of sleep disorders (e.g., melatonin and melatonin receptor agonists) and, optionally, at least one pharmaceutically acceptable carrier or excipient.

A further aspect of the invention relates to a pharmaceutical composition comprising a therapeutically effective amount of a 1-amino-alkylcyclohexane derivative (e.g., neramexane or a pharmaceutically acceptable salt thereof such as neramexane mesylate) in combination with other therapies for sleep disorders and, optionally, at least one pharmaceutically acceptable carrier or excipient.

### DETAILED DESCRIPTION OF THE INVENTION

As used herein, the term tinnitus includes all manifestations of subjective and objective tinnitus as well as acute, subacute and chronic forms.

As used herein, the term sleep disorders includes insomnia, (i.e., disorders related to initiating and maintaining sleep), disorders of the sleep-wake cycle, dysfunctions associated with sleep, sleep stages, or partial arousals (parasomnias), disorders of excessive somnolence, and non-restorative sleep.

As used herein, the term neurological diseases includes neuropathic and chronic pain, fibromyalgia, periodic limb movement, restless-legs-syndrome, and chronic fatigue syndrome.

The term 1-amino-alkylcyclohexane derivative is used herein to describe a compound which is a 1-amino-alkylcyclohexane or a compound derived from 1-amino-alkylcyclohexane (or an available derivative thereof, such as neramexane) in the process used to create a similar but slightly different drug. The present 1-amino-alkylcyclohexane derivatives may also be described as “1-aminocyclohexane derivatives.”

The 1-amino-alkylcyclohexane derivatives of the present invention may be represented by the general formula (I):

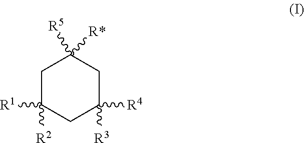

wherein R* is —(CH₂)ₙ—(CR⁶R⁷)ₘ—NR⁸R⁹
wherein n+m=0, 1, or 2
wherein R¹ through R⁷ are independently selected from the group consisting of hydrogen and C₁₋₆alkyl, wherein R⁸ and R⁹ are independently selected from the group consisting of hydrogen and C₁₋₆alkyl or together represent lower-alkylene —(CH₂)ₓ— wherein x is 2 to 5, inclusive, and optical isomers, enantiomers, hydrates, and pharmaceutically-acceptable salts thereof.

Non-limiting examples of the 1-amino-alkylcyclohexanes used according to the present invention include:
1-amino-1,3,5-trimethylcyclohexane,
1-amino-1(trans),3(trans),5-trimethylcyclohexane,
1-amino-1(cis),3(cis),5-trimethylcyclohexane,
1-amino-1,3,3,5-tetramethylcyclohexane,
1-amino-1,3,3,5,5-pentamethylcyclohexane (neramexane),
1-amino-1,3,5,5-tetramethyl-3-ethylcyclohexane,
1-amino-1,5,5-trimethyl-3,3-diethylcyclohexane,
1-amino-1,5,5-trimethyl-cis-3-ethylcyclohexane,
1-amino-(1S,5S)cis-3-ethyl-1,5,5-trimethylcyclohexane,
1-amino-1,5,5-trimethyl-trans-3-ethylcyclohexane,
1-amino-(1R,5S)trans-3-ethyl-1,5,5-trimethylcyclohexane,
1-amino-1-ethyl-3,3,5,5-tetramethylcyclohexane,
1-amino-1-propyl-3,3,5,5-tetramethylcyclohexane,
N-methyl-1-amino-1,3,3,5,5-pentamethylcyclohexane,
N-ethyl-1-amino-1,3,3,5,5-pentamethyl-cyclohexane,
N-(1,3,3,5,5-pentamethylcyclohexyl)pyrrolidine,
3,3,5,5-tetramethylcyclohexylmethylamine,
1-amino-1-propyl-3,3,5,5-tetramethylcyclohexane,
1amino-1,3,3,5(trans)-tetramethylcyclohexane (axial amino group),
3-propyl-1,3,5,5-tetramethylcyclohexylamine semihydrate,
1-amino-1,3,5,5-tetramethyl-3-ethylcyclohexane,
1-amino-1,3,5-trimethylcyclohexane,
1-amino-1,3-dimethyl-3-propylcyclohexane,
1-amino-1,3(trans),5(trans)-trimethyl-3(cis)-propylcyclohexane,
1-amino-1,3-dimethyl-3-ethylcyclohexane,
1-amino-1,3,3-trimethylcyclohexane,
cis-3-ethyl-1(trans)-3(trans)-5-trimethylcyclohexamine,
1-amino-1,3(trans)-dimethylcyclohexane,
1,3,3-trimethyl-5,5-dipropylcyclohexylamine,
1-amino-1-methyl-3(trans)-propylcyclohexane,
1-methyl-3(cis)-propylcyclohexylamine,
1-amino-1-methyl-3(trans)-ethylcyclohexane,
1-amino-1,3,3-trimethyl-5(cis)-ethylcyclohexane,
1-amino-1,3,3-trimethyl-5(trans)-ethylcyclohexane,
cis-3-propyl-1,5,5-trimethylcyclohexylamine,
trans-3-propyl-1,5,5-trimethylcyclohexylamine,
N-ethyl-1,3,3,5,5-pentamethylcyclohexylamine,
N-methyl-1-amino-1,3,3,5.5-pentamethylcyclohexane,
1-amino-1-methylcyclohexane,
N,N-dimethyl-1-amino-1,3,3,5,5-pentamethylcyclohexane,
2-(3,3,5,5-tetramethylcyclohexyl)ethylamine,
2-methyl-1-(3,3,5,5-tetramethylcyclohexyl)propyl-2-amine,
2-(1,3,3,5,5-pentamethylcyclohexyl)-ethylamine semihydrate,
N-(1,3,3,5,5-pentamethylcyclohexyl)-pyrrolidine,
1-amino-1,3(trans),5(trans)-trimethylcyclohexane,
1-amino-1,3(cis),5(cis)-trimethylcyclohexane,
1-amino-(1R,5S)trans-5-ethyl-1,3,3-trimethylcyclohexane,
1-amino-(1S,5S)cis-5-ethyl-1,3,3-trimethylcyclohexane,
1-amino-1,5,5-trimethyl-3(cis)-isopropyl-cyclohexane,
1-amino-1,5,5-trimethyl-3(trans)-isopropyl-cyclohexane,
1-amino-1-methyl-3(cis)-ethyl-cyclohexane,
1-amino-1-methyl-3(cis)-methyl-cyclohexane,
1-amino-5,5-diethyl-1,3,3-trimethyl-cyclohexane,
1-amino-1,3,3,5,5-pentamethylcyclohexane,
1-amino-1,5,5-trimethyl-3,3-diethylcyclohexane,
1-amino-1-ethyl-3,3,5,5-tetramethylcyclohexane,
N-ethyl-1-amino-1,3,3,5,5-pentamethylcyclohexane,
N-(1,3,5-trimethylcyclohexyl)pyrrolidine or piperidine,
N-[1,3(trans),5(trans)-trimethylcyclohexyl]pyrrolidine or piperidine,
N-[1,3(cis),5(cis)-trimethylcyclohexyl]pyrrolidine or piperidine,
N-(1,3,3,5-tetramethylcyclohexyl)pyrrolidine or piperidine,
N-(1,3,3,5,5-pentamethylcyclohexyl)pyrrolidine or piperidine,
N-(1,3,5,5-tetramethyl-3-ethylcyclohexyl)pyrrolidine or piperidine,
N-(1,5,5-trimethyl-3,3-diethylcyclohexyl)pyrrolidine or piperidine,
N-(1,3,3-trimethyl-cis-5-ethylcyclohexyl)pyrrolidine or piperidine,
N-[(1S,5S)cis-5-ethyl-1,3,3-trimethylcyclohexyl]pyrrolidine or piperidine,
N-(1,3,3-trimethyl-trans-5-ethylcyclohexyl)pyrrolidine or piperidine,
N-[(1R,5S)trans-5-ethyl,3,3-trimethylcyclohexyl]pyrrolidine or piperidine,
N-(1-ethyl-3,3,5,5-tetramethylyclohexyl)pyrrolidine or piperidine,
N-(1-propyl-3,3,5,5-tetramethylcyclohexyl)pyrrolidine or piperidine,
N-(1,3,3,5,5-pentamethylcyclohexyl)pyrrolidine,
   and optical isomers, diastereomers, enantiomers, hydrates, their pharmaceutically acceptable salts, and mixtures thereof.

1-Amino-alkylcyclohexane derivatives (e.g., neramexane, 1-amino-1,3,3,5,5-pentamethylcyclohexane) are disclosed in U.S. Pat. Nos. 6,034,134 and 6,071,966. 1-Amino-alkylcyclohexane derivatives (e.g., neramexane) may be used according to the invention in the form of any of pharmaceutically acceptable salts, solvates, isomers, conjugates, and prodrugs, any references to 1-amino-alkylcyclohexane derivatives (e.g., neramexane) in this description should be understood as also referring to such salts, solvates, isomers, conjugates, and prodrugs.

As used herein the term melatonin receptor agonist is one of a group of substances known in the art to modulate melatonin receptors, such substances including ramelteon (a MT1 and MT2 agonist) or agomelatine (a combined melatonin receptor agonist and serotonin receptor antagonist) and pharmaceutically acceptable salts thereof.

Pharmaceutically acceptable salts include, but are not limited to, acid addition salts, such as those made with hydrochloric, methylsulfonic, hydrobromic, hydroiodic, perchloric, sulfuric, nitric, phosphoric, acetic, propionic, glycolic, lactic, pyruvic, malonic, succinic, fumaric, tartaric, citric, benzoic, carbonic, cinnamic, mandelic, methanesulfonic, ethanesulfonic, hydroxyethanesulfonic, benezenesulfonic, p-toluene sulfonic, cyclohexanesulfamic, salicyclic, p-aminosalicylic, 2-phenoxybenzoic, and 2-acetoxybenzoic acid. All of these salts (or other similar salts) may be prepared by conventional means. The nature of the salt is not critical, provided that it is non-toxic and does not substantially interfere with the desired pharmacological activity.

The term “analog” or “derivative” is used herein in the conventional pharmaceutical sense, to refer to a molecule that structurally resembles a reference molecule (such as neramexane), but has been modified in a targeted and controlled manner to replace one or more specific substituents of the referent molecule with an alternate substituent, thereby generating a molecule which is structurally similar to the reference molecule. Synthesis and screening of analogs (e.g., using structural and/or biochemical analysis), to identify slightly modified versions of a known compound which may have improved or biased traits (such as higher potency and/or selectivity at a specific targeted receptor type, greater ability to penetrate mammalian blood-brain barriers, fewer side effects, etc.) is a drug design approach that is well known in pharmaceutical chemistry.

The term “treat” is used herein to mean to relieve or alleviate at least one symptom of a disease in a subject. Within the meaning of the present invention, the term “treat” also denotes to arrest, delay the onset (i.e., the period prior to clinical manifestation of a disease) and/or reduce the risk of developing or worsening a disease.

The term “therapeutically effective” applied to dose or amount refers to that quantity of a compound or pharmaceutical composition that is sufficient to result in a desired activity upon administration to a mammal in need thereof.

The phrase “pharmaceutically acceptable”, as used in connection with compositions of the invention, refers to molecular entities and other ingredients of such compositions that are physiologically tolerable and do not typically produce untoward reactions when administered to a mammal (e.g., human). The term “pharmaceutically acceptable” may also mean approved by a regulatory agency of the Federal or a state government or listed in the U.S. Pharmacopeia or other generally recognized pharmacopeia for use in mammals, and more particularly in humans.

The term “carrier” applied to pharmaceutical compositions of the invention refers to a diluent, excipient, or vehicle with which an active compound (e.g., neramexane) is administered. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Suitable pharmaceutical carriers are described in “Remington's Pharmaceutical Sciences” by A. R. Gennaro, 20^{th} Edition.

The term “about” or “approximately” usually means within 20%, alternatively within 10%, including within 5% of a given value or range. Alternatively, especially in biological systems, the term “about” means within about a log (i.e., an order of magnitude), including within a factor of two of a given value.

In conjunction with the methods of the present invention, also provided are pharmaceutical compositions comprising a therapeutically effective amount of a 1-amino-alkylcyclohexane derivative (e.g., neramexane). The compositions of the invention may further comprise a carrier or excipient (all pharmaceutically acceptable). The compositions may be formulated for once-a-day administration, twice-a-day administration, or three times a day administration.

The active ingredient (e.g., neramexane, such as neramexane mesylate) or the composition of the present invention may be used for the treatment of at least one of the mentioned disorders, wherein the medicament is adapted to or appropriately prepared for a specific administration as disclosed herein (e.g., to once-a-day, twice-a-day administration, or three times a day administration). For this purpose the package leaflet and/or the patient information contains corresponding information.

The active ingredient (e.g., neramexane, such as neramexane mesylate) or the composition of the present invention may be used for the manufacture of a medicament for the treatment of at least one of the mentioned disorders, wherein the medicament is adapted to or appropriately prepared for a specific administration as disclosed herein (e.g., to once-a-day, twice-a-day administration, or three times a day administration). For this purpose the package leaflet and/or the patient information contains corresponding information.

According to the present invention, the dosage form of the 1-amino-alkylcyclohexane derivative (e.g., neramexane) may be a solid, semisolid, or liquid formulation according to the following.

The 1-amino-alkylcyclohexane derivatives of the present invention (e.g., neramexane) may be administered orally, topically, parenterally, or mucosally (e.g., buccally, by inhalation, or rectally) in dosage unit formulations containing conventional non-toxic pharmaceutically acceptable carriers. In another embodiment for administration to pediatric subjects, the 1-amino-alkylcyclohexane derivative may be formulated as a flavored liquid (e.g., peppermint flavor). The 1-amino-alkylcyclohexane derivatives of the present invention may be administered orally in the form of a capsule, a tablet, or the like, or as a semi-solid, or liquid formulation (see Remington's Pharmaceutical Sciences, 20^{th} Edition, by A. R. Gennaro).

For oral administration in the form of a tablet or capsule, the 1-amino-alkylcyclohexane derivatives of the present invention (e.g., neramexane) may be combined with non-toxic, pharmaceutically acceptable excipients such as binding agents (e.g., pregelatinized maize starch, polyvinylpyrrolidone or hydroxypropyl methylcellulose); fillers (e.g., lactose, sucrose, glucose, mannitol, sorbitol and other reducing and non-reducing sugars, microcrystalline cellulose, calcium sulfate, or calcium hydrogen phosphate); lubricants (e.g., magnesium stearate, talc, or silica, steric acid, sodium stearyl fumarate, glyceryl behenate, calcium stearate, and the like); disintegrants (e.g., potato starch or sodium starch glycolate); or wetting agents (e.g., sodium lauryl sulphate), coloring and flavoring agents, gelatin, sweeteners, natural and synthetic gums (such as acacia, tragacanth or alginates), buffer salts, carboxymethylcellulose, polyethyleneglycol, waxes, and the like.

The tablets may be coated with a concentrated sugar solution which may contain e.g., gum arabic, gelatine, talcum, titanium dioxide, and the like. Alternatively, the tablets can be coated with a polymer that dissolves in a readily volatile organic solvent or mixture of organic solvents. In specific embodiments, neramexane is formulated in immediate-release (IR) or modified-release (MR) tablets. Immediate release solid dosage forms permit the release of most or all of the active ingredient over a short period of time, such as 60 minutes or less, and make rapid absorption of the drug possible (immediate release formulations of 1-amino-alkylcyclohexanes such as neramexane are disclosed in US Published Application Nos. 2006/0002999 and 2006/0198884, the subject matter of which is hereby incorporated by reference). Modified release solid oral dosage forms permit the sustained release of the active ingredient over an extended period of time in an effort to maintain therapeutically effective plasma levels over similarly extended time intervals and/or to modify other pharmacokinetic properties of the active ingredient (modified release formulations of neramexane are disclosed in US Published Application No. 2007/0141148, the subject matter of which is hereby incorporated by reference). For example, neramexane mesylate may be formulated in a modified release dosage form (including modified release tablets) to provide a 50 mg dose of neramexane mesylate.

For the formulation of soft gelatin capsules, the 1-amino-alkylcyclohexane derivatives of the present invention (e.g., neramexane) may be admixed with e.g., a vegetable oil or poly-ethylene glycol. Hard gelatin capsules may contain granules of the active substances using either the above mentioned excipients for tablets e.g., lactose, saccharose, sorbitol, mannitol, starches (e.g., potato starch, corn starch or amylopectin), cellulose derivatives or gelatine. Also liquids or semisolids of the drug can be filled into hard gelatine capsules.

The 1-amino-alkylcyclohexane derivatives of the present invention (e.g., neramexane) can also be introduced in microspheres or microcapsules, e.g., fabricated from polyglycolic acid/lactic acid (PGLA) (see, e.g., U.S. Pat. Nos. 5,814,344; 5,100,669 and 4,849,222; PCT Publications No. WO 95/11010 and WO 93/07861). Biocompatible polymers may be used in achieving controlled release of a drug, include for example, polylactic acid, polyglycolic acid, copolymers of polylactic and polyglycolic acid, polyepsilon caprolactone, polyhydroxybutyric acid, polyorthoesters, polyacetals, polyhydropyrans, polycyanoacrylates, and cross-linked or amphipathic block copolymers of hydrogels.

Formulation of the 1-amino-alkylcyclohexane derivatives of the present invention in a semi-solid or liquid form may also be used. The 1-amino-alkylcyclohexane derivative (e.g., neramexane) may constitute between 0.1 and 99% by weight of the formulation, more specifically between 0.5 and 20% by weight for formulations intended for injection and between 0.2 and 50% by weight for formulations suitable for oral administration.

In one embodiment of the invention, the 1-amino-alkylcyclohexane derivative (e.g., neramexane) is administered in a modified release formulation. Modified release dosage forms provide a means for improving patient compliance and for ensuring effective and safe therapy by reducing the incidence of adverse drug reactions. Compared to immediate release dosage forms, modified release dosage forms can be used to prolong pharmacologic action after administration, and to reduce variability in the plasma concentration of a drug throughout the dosage interval, thereby eliminating or reducing sharp peaks.

A modified release form dosage may comprise a core either coated with or containing a drug. The core being is then coated with a release modifying polymer within which the drug is dispersed. The release modifying polymer disintegrates gradually, releasing the drug over time. Thus, the outer-most layer of the composition effectively slows down and thereby regulates the diffusion of the drug across the coating layer when the composition is exposed to an aqueous environment, i.e. the gastrointestinal tract. The net rate of diffusion of the drug is mainly dependent on the ability of the gastric fluid to penetrate the coating layer or matrix and on the solubility of the drug itself.

In another embodiment of the invention, the 1-amino-alkylcyclohexane derivative (e.g., neramexane) is formulated in an oral, liquid formulation. Liquid preparations for oral administration can take the form of, for example, solutions, syrups, emulsions or suspensions, or they can be presented as a dry product for reconstitution with water or other suitable vehicle before use. Preparations for oral administration can be suitably formulated to give controlled or postponed release of the active compound. Oral liquid formulations of 1-amino-alkylcyclohexanes, such as neramexane, are described in PCT International Application No. PCT/US2004/037026, the subject matter of which is hereby incorporated by reference.

For oral administration in liquid form, 1-amino-alkylcyclohexane derivatives of the present invention (e.g., neramexane) may be combined with non-toxic, pharmaceutically acceptable inert carriers (e.g., ethanol, glycerol, water), suspending agents (e.g., sorbitol syrup, cellulose derivatives or hydrogenated edible fats), emulsifying agents (e.g., lecithin or acacia), non-aqueous vehicles (e.g., almond oil, oily esters, ethyl alcohol or fractionated vegetable oils), preservatives (e.g., methyl or propyl-p-hydroxybenzoates or sorbic acid), and the like. Stabilizing agents such as antioxidants (BHA, BHT, propyl gallate, sodium ascorbate, citric acid) can also be added to stabilize the dosage forms. For example, solutions may contain from about 0.2% to about 20% by weight of neramexane, with the balance being sugar and mixture of ethanol, water, glycerol and propylene glycol. Optionally, such liquid formulations may contain coloring agents, flavoring agents, saccharine and carboxymethyl-cellulose as a thickening agent or other excipients.

In another embodiment, a therapeutically effective amount of a 1-amino-alkylcyclohexane derivative (e.g., neramexane) is administered in an oral solution containing a preservative, a sweetener, a solubilizer, and a solvent. The oral solution may include one or more buffers, flavorings, or additional excipients. In a further embodiment, a peppermint or other flavoring is added to the neramexane derivative oral liquid formulation.

For administration by inhalation, 1-amino-alkylcyclohexane derivatives (e.g., neramexane) of the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or other suitable gas. In the case of a pressurized aerosol, the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

Solutions for parenteral applications by injection may be prepared in an aqueous solution of a water-soluble pharmaceutically acceptable salt of the active substances, for example in a concentration of from about 0.5% to about 10% by weight. These solutions may also contain stabilizing agents and/or buffering agents and may conveniently be provided in various dosage unit ampoules.

The formulations of the invention may be delivered parenterally, i.e., by intravenous (i.v.), intracerebroventricular (i.c.v.), subcutaneous (s.c.), intraperitoneal (i.p.), intramuscular (i.m.), subdermal (s.d.), or intradermal (i.d.) administration, by direct injection, via, for example, bolus injection or continuous infusion. Formulations for injection can be presented in unit dosage form, e.g., in ampoules or in multi-dose containers, with an added preservative. Alternatively, the active ingredient may be in powder form for reconstitution with a suitable vehicle, e.g., sterile pyrogen-free water, before use.

The invention also provides a pharmaceutical pack or kit comprising one or more containers containing a 1-amino-alkylcyclohexane derivative (e.g., neramexane) and, optionally, more of the ingredients of the formulation. In a specific embodiment, neramexane is provided as an oral solution (2 mg/ml) for administration with the use of a 2 teaspoon capacity syringe (dosage KORC®). Each oral syringe has hatch marks for measurement, with lines on the right side of the syringe (tip down) representing tsp units, and those on the left representing ml units.

The optimal therapeutically effective amount may be determined experimentally, taking into consideration the exact mode of administration, from in which the drug is administered, the indication toward which the administration is directed, the subject involved (e.g., body weight, health, age, sex, etc.), and the preference and experience of the physician or veterinarian in charge.

Dosage units for rectal application may be solutions or suspensions or may be prepared in the form of suppositories or retention enemas comprising neramexane in a mixture with a neutral fatty base, or gelatin rectal capsules comprising the active substances in admixture with vegetable oil or paraffin oil.

Toxicity and therapeutic efficacy of the compositions of the invention may be determined by standard pharmaceutical procedures in experimental animals, e.g., by determining the LD₅₀ (the dose lethal to 50% of the population) and the ED₅₀ (the dose therapeutically effective in 50% of the population). The dose ratio between therapeutic and toxic effects is the therapeutic index and it may be expressed as the ratio LD₅₀/ED₅₀. Compositions that exhibit large therapeutic indices are preferred.

Suitable daily doses of the active compounds of the invention in therapeutic treatment of humans are about 0.01-10 mg/kg bodyweight on peroral administration and 0.001-10 mg/kg bodyweight on parenteral administration. For example, for adults, suitable daily doses of neramexane (e.g. neramexane mesylate) are within the range from about 5 mg to about 150 mg per day, such as from about 5 mg to about 120 mg, from about 5 mg to about 100 mg, or from about 5 mg to about 75 mg, or from about 5 mg to about 50 mg, such as 25 mg or 37.5 mg or 50 mg, per day. For example the daily dose may be body weight-adjusted such as 50 mg/day up to 90 kg body weight or 75 mg/day for patients with a body weight of ≧90 kg. An equimolar amount of another pharmaceutically acceptable salt, a solvate, an isomer, a conjugate, a prodrug or a derivative thereof, such as neramexane hydrochloride, is also suitable. For pediatric subjects aged 4-14, neramexane (e.g. neramexane mesylate) may be administered as an oral, liquid dosage form, at about 0.5 mg/day, up to a maximum dose of 10 mg/day.

The daily doses indicated herein may be administered, for example, as one or two dosing units once, twice or three times per day. Suitable doses per dosage unit may therefore be the daily dose divided (for example, equally) between the number of dosage units administered per day, and will thus typically be about equal to the daily dose or one half, one third, one quarter or one sixth thereof. Dosages per dosage unit may thus be calculated from each daily dosage indicated herein. A daily dose of 5 mg, for example may be seen as providing a dose per dosage unit of, for example, about 5 mg, 2.5 mg, 1.67 mg, 1.25 mg and 0.83 mg, depending upon the dosing regimen chosen. Correspondingly, a dosage of 150 mg per day corresponds to dosages per dosing unit of, for example, about 150 mg, 75 mg, 50 mg, 37.5 mg, and 25 mg for corresponding dosing regimens.

Treatment duration may be short-term, e.g., several weeks (for example 8-14 weeks), or long-term until the attending physician deems further administration no longer is necessary.

The 1-amino-alkylcyclohexane derivatives of the present invention (e.g., neramexane) may be administered as a monotherapy, or in combination with another agent prescribed for the treatment of sleep disorders.

The term “combination” applied to active ingredients is used herein to define a single pharmaceutical composition (formulation) comprising two active agents (e.g., a pharmaceutical composition comprising a 1-amino-alkylcyclohexane derivative, such as neramexane, and another agent prescribed for the treatment of sleep disorders) or two separate pharmaceutical compositions, each comprising an active agent (e.g. a pharmaceutical composition comprising a 1-amino-alkylcyclohexane derivative, such as neramexane, or another agent prescribed for the treatment of sleep disorders), to be administered conjointly.

Within the meaning of the present invention, the term “conjoint administration” is used to refer to administration of 1-amino-alkylcyclohexane derivative, such as neramexane, and a second active agent (e.g. another agent prescribed for the treatment of sleep disorders) simultaneously in one composition, or simultaneously in different compositions, or sequentially. For the sequential administration to be considered “conjoint”, however, 1-amino-alkylcyclohexane derivative, such as neramexane, and the second active agent must be administered separated by a time interval which still permits the resultant beneficial effect for treating sleep disorders associated with tinnitus and/or neurological diseases in a mammal.

### EXAMPLES OF REPRESENTATIVE FORMULATIONS

With the aid of commonly used solvents, auxiliary agents and carriers, active ingredients may be processed into tablets, coated tablets, capsules, drip solutions, suppositories, injection and infusion preparations, and the like and can be therapeutically applied by the oral, rectal, parenteral, and additional routes. Tablets suitable for oral administration may be prepared by conventional tabletting techniques. The following example is given by way of illustration only and is not to be construed as limiting.

### Formulation Example 1

### Neramexane Mesylate Immediate Release Tablets

The following tables provide the make-up of neramexane immediate release tablets in 12.5, 25.0, 37.5, and 50.0 mg dosages, including active components, coating agents, and other excipients.

TABLE 1
Neramexane mesylate, 12.5 mg film coated tablets

| | Amount | |
|---|---|---|
| Component | [mg] | Function |
| Neramexane mesylate | 12.50 | Active |
| | | pharmaceutical |
| | | ingredient |
| Cellulose microcrystalline | 103.25 | Binder |
| Croscarmellose sodium | 6.25 | Disintegrant |
| Silicon dioxide, colloidal | 1.25 | Flow promoter |
| Talc | 1.25 | Glident |
| Magnesium stearate | 0.50 | Lubricant |
| core weight | 125.00 | |
| Coating (HPMC), Opadry or | 5.00 | Coating |
| Sepifilm | | |
| Coat weight | 5.00 | |
| coated tablet total weight | 130.00 | |

TABLE 2
Neramexane mesylate, 25.0 mg film coated tablets

| | Amount | |
|---|---|---|
| Component | [mg] | Function |
| Neramexane mesylate | 25.00 | Active |
| | | pharmaceutical |
| | | ingredient |
| Cellulose microcrystalline | 206.50 | Binder |
| Croscarmellose sodium | 12.5 | Disintegrant |
| Silicon dioxide, colloidal | 2.50 | Flow promoter |
| Talc | 2.50 | Glident |
| Magnesium stearate | 1.00 | Lubricant |
| core weight | 250.00 | |
| Coating (HPMC), Opadry or | 10.00 | Coating |
| Sepifilm | | |
| Coat weight | 10.00 | |
| coated tablet total weight | 260.00 | |

TABLE 3
Neramexane mesylate, 37.5 mg film coated tablets

| | Amount | |
|---|---|---|
| Component | [mg] | Function |
| Neramexane mesylate | 37.50 | Active |
| | | pharmaceutical |
| | | ingredient |
| Cellulose microcrystalline | 309.75 | Binder |
| Croscarmellose sodium | 18.75 | Disintegrant |
| Silicon dioxide, colloidal | 3.75 | Flow promoter |
| Talc | 3.75 | Glident |
| Magnesium stearate | 1.50 | Lubricant |
| core weight | 375.00 | |
| Coating (HPMC), Opadry or | 15.00 | Coating |
| Sepifilm | | |
| Coat weight | 15.00 | |
| coated tablet total weight | 390.00 | |

TABLE 4
Neramexane mesylate, 50.0 mg film coated tablets

| | Amount | |
|---|---|---|
| Component | [mg] | Function |
| Neramexane mesylate | 50.00 | Active |
| | | pharmaceutical |
| | | ingredient |
| Cellulose microcrystalline | 413.00 | Binder |
| Croscarmellose sodium | 25.00 | Disintegrant |
| Silicon dioxide, colloidal | 5.00 | Flow promoter |
| Talc | 5.00 | Glident |
| Magnesium stearate | 2.00 | Lubricant |
| core weight | 500.00 | |
| Coating (HPMC), Opadry or | 20.00 | Coating |
| Sepifilm | | |
| Coat weight | 20.00 | |
| coated tablet total weight | 520.00 | |

### EXAMPLES

The following examples illustrate the invention without limiting its scope.

### Example 1

### Double Blind Placebo Controlled Pilot Trial of Neramexane for Treatment of Tinnitus

The objective of this pilot project was to conduct a clinical trial to assess the efficacy of neramexane as a treatment for tinnitus. The primary objective of this study was to compare the efficacy, tolerability and safety of neramexane mesylate at three different dosages (25, 50 or 75 mg/d) with placebo in subjects with subjective tinnitus of at least moderate severity.

### Study Design

In a double-blind, multicenter, randomized, placebo-controlled, parallel-group study, the efficacy of neramexane in subjects suffering from tinnitus of at least moderate severity was assessed. Approximately 100 patients, who fulfilled particular inclusion criteria and met none of particular exclusion criteria, were randomized to each of four double-blind treatment groups (neramexane mesylate 25, 50, 75 mg/d or placebo), resulting in approximately 400 patients in total.

The double-blind, 16-week treatment period consisted of a 4-week uptitration period and a 12-week fixed-dose treatment period at unchanged maintenance b.i.d. dosing. In case of poor tolerability, however, the investigator could consider a dose reduction by 25 mg/d (or placebo, respectively). After the treatment phase, there was a 4-week follow-up period with no active treatment and concomitant therapy restrictions. In total, this study involved seven study visits: screening, baseline, and at the end of weeks 4, 8, 12, 16, and 20.

The scheduled visits for evaluation of each patient were as follows:

Visit 1 (screening): After signing the consent form, the subject underwent a physical examination and clinical laboratory testing. Patient eligibility for the study was evaluated via a check of inclusion/exclusion criteria. An initial Tinnitus Interview was conducted. The subject also completed a Tinnitus-Beeintrachtigungs-Fragebogen (TBF-12) (i.e., a 12-item German modified and validated version (Greimel K V et al., Tinnitus-Beeinträchtigungs-Fragebogen (TBF-12). Manual. Frankfurt am Main: Swets & Zeitlinger B. V.; 2000) of the 25-item Tinnitus Handicap Inventory or THI (Newman C W, et al. Development of the Tinnitus Handicap Inven-tory. Arch Otolaryngol Head Neck Surg 1996; 122(2): 143-148; Newman C W, et al. Psychometric adequacy of the Tinnitus Handicap Inventory (THI) for evaluating treatment outcome. J Am Acad Audiol 1998; 9(2): 153-160.)), a Hospital Anxiety and Depression Scale—Depression Subscale (HADS-D)

Questionnaire and a Hyperacusis (Geräuschüberempfindlichkeit-Fragenbogen (GÜF)) Questionnaire (if applicable).

Visit 2 (baseline): The subject was asked about adverse events and changes in concomitant medication/disease, which events/changes were documented. The subject was evaluated for study eligibility based on a review of the inclusion/exclusion criteria. Trial procedures as well as allowed and forbidden concomitant medications were reviewed with the subject. An initial Tinnitus Interview was conducted. The subject also completed a TBF-12, HADS-D Questionnaire and GÜF Questionnaire (if applicable). The subject was enrolled in the study and study medication (placebo or neramexane) was dispensed as described below.

Visit 3 (Week 4): This visit occurred at the end of the 4-week up-titration sequence. The subject was asked about adverse events and changes in concomitant medication/disease, which events/changes were documented. A follow-up Tinnitus Interview was conducted. The subject also completed a TBF-12, HADS-D Questionnaire and GÜF Questionnaire (if applicable). Medication compliance was assessed, and medication for the next 4 weeks was dispensed as described below.

Visit 4 (Week 8): This visit occurred at the end of the first 4-week fixed-dose double-blind treatment period. The subject was asked about adverse events and changes in concomitant medication/disease, which changes are documented. Blood samples were collected in order to determine neramexane pre-dose concentration. A follow-up Tinnitus Interview was conducted. The subject also completed a TBF-12, HADS-D Questionnaire and GÜF Questionnaire (if applicable). Medication compliance was assessed and, medication for the next 4 weeks was dispensed as described below.

Visit 5 (Week 12): This visit occurred at the end of the second 4-week fixed-dose double-blind treatment period. The subject was asked about adverse events and changes in concomitant medication/disease, which changes are documented. A follow-up Tinnitus Interview was conducted. The subject also completed a TBF-12, HADS-D Questionnaire and GÜF Questionnaire (if applicable). Medication compliance was assessed and, medication for the next 4 weeks was dispensed as described below.

Visit 6 (Week 16, end of treatment). This visit occurred at the end of the 12-week fixed-dose double-blind treatment period. The subject was asked about adverse events and changes in concomitant medication/disease, which changes are documented. A clinical laboratory evaluation was performed. A follow-up Tinnitus Interview was conducted, and the subject completed a TBF-12, HADS-D Questionnaire and GÜF Questionnaire (if applicable). Pure-tone audiometry (air conduction) was also conducted.

Visit 7 (Week 20): This visit occurred at the end of the 4-week follow-up period after the last study medication dose. Review of concomitant medications as well as the occurrence of adverse events since the last visit is conducted with subject. A follow-up Tinnitus Interview was conducted, and the subject completed a TBF-12, HADS-D Questionnaire and GÜF Questionnaire (if applicable).

### Administration of Neramexane

Neramexane mesylate immediate release tablets (12.5 mg and 25 mg) and matching placebo tablets were administered as film coated tablets.

Medication was supplied in blister boxes that were dispensed from Visit 2 to Visit 5. Each blister box contained 4 blister cards for 4 treatment weeks and 1 blister card as reserve. Blister cards were identified by treatment weeks. Daily medication within the blister cards were identified per day. Study medication for each study day consisted of 4 separate tablets. One blister card contained of 32 tablets (7×4 tablets, 4 tablets per day, and a reserve of 4 tablets for one day). One package of medication per patient consisted of 5 boxes. Box 2 was added as reserve medication for box 1 (uptitration period) and was only to be dispensed if the subject lost a blister card of box 1 or the whole box.

Study medication was dispensed at Visit 2 (baseline, day, 0). Each patient received one blister box containing 5 blister cards (including one reserve blister) of double-blind study medication (i.e., 32 tablets). Subjects were instructed to take 2 tablets twice daily (4 tablets/d), beginning the day after dispensing of the study medication, until they returned for their next study visit (Visit 3). For those subjects assigned to receive active medication, some placebo tablets were incorporated into the dosing regimen to ensure blinding during the uptitration period. The target fixed-maintenance dose of 25, 50, or 75 mg neramexane mesylate/d was administered starting with the fifth week of double-blind treatment and was continued throughout the study. At each of the subsequent visits (Visits 3, 4, and 5, corresponding to end of week 4, 8 and 12) patients received another blister box containing 5 blister cards for the 4 week intervals, with double-blind medication for the intervening treatment period until the next study visit. The dosing schedule is shown in Table 5.

Throughout the double-blind treatment period, patients were to continue to take 2×2 tablets of medication daily at a constant interval of 12 hours. In case the patient had already taken the morning dose of study medication on the day of Visits 4 and 6 (Week 8 and Week 16), no scheduled blood sampling was to be done. The investigator had to re-dispense a sufficient amount of study medication. The patient should continue to take 2 by 2 tablets at a constant interval of 12 hours and had return for pre-dose Neramexane blood sampling within the time window of Visits 4 and 6.

TABLE 5
Administration of Neramexane mesylate

| | | | | | 12-week fixed-dose | | 4-week |
|---|---|---|---|---|---|---|---|
| | 4-week double-blind up-titration period | | | | double-blind period | | follow-up |
| Treatment group | Week 1 | Week 2 | Week 3 | Week 4 | Weeks 5-16 | | Weeks 17-20 |
| High-dose | 12.5/0 | 12.5/12.5 | 25/12.5 | 25/25 | 37.5/37.5 | (75 mg/d) | — |
| Medium-dose | 12.5/0 | 12.5/0 | 12.5/12.5 | 25/12.5 | 25/25 | (50 mg/d) | — |
| Low-dose | 12.5/0 | 12.5/0 | 12.5/0 | 12.5/0 | 12.5/12.5 | (25 mg/d) | — |

| | | | | | | |
|---|---|---|---|---|---|---|
| Placebo | 0/0 | 0/0 | 0/0 | 0/0 | 0/0 | — |

xx/xx refers to the morning/evening dose in mg, respectively

In case of poor tolerability the investigator could consider a dose reduction of 25 mg/d by omitting the bigger tablet in the morning which constituted an effective dose reduction only in the 75 mg/d and 50 mg/d neramexane mesylate groups. After omitting the bigger tablet (25 mg or placebo, respectively) of the morning dose, these patients could then continue the course of the study as scheduled, while receiving only one smaller tablet as the morning dose (12.5 mg or placebo, respectively) and 2 tablets of different sizes (12.5 mg, 25 mg or placebo, respectively) as the evening dose. The dose was to be kept stable until the end of the study.

Subjects were instructed to take study medication always at an individually convenient, but stable time point throughout the study course and at a constant dosing interval of 12 hours whenever possible (e.g. 6:00 h and 18:00 h or 8:00 h and 20:00 h). At each study visit, the investigator enquired the time points of study medication intake on the preceding day. At the end of week 4, 8, 12, and 16 (or upon early termination), patients returned to the study site bringing their blister boxes containing 5 blister cards with them for an assessment of medication compliance.

### Efficacy

### Primary Outcome

The change in TBF-12 total score from baseline (Visit 2) to the endpoint visit (Visit 6, i.e. Week 16) was the primary efficacy endpoint in this study.

### Secondary Outcomes

TBF-12 total score (values and absolute change from baseline) at all post-baseline visits except the endpoint visit.

Change in the TBF-12 total score from Week 16 to Week 20 (values and absolute changes).

TBF-12 factorial scores (values and absolute change from baseline, including the change from Week 16 to Week 20) at all post-baseline visits. Hyperacusis questionnaire GÜF (“Geräuschüberempfindlichkeits-Fragebogen”), values and absolute change from baseline, including the change from Week 16 to Week 20, total and factorial scores at all post-baseline visits if hyperacusis was present.

Clinical global impression of change: item 27 of the tinnitus follow-up interview was summarized after dichotomization of the responses in any improvement (values 1, 2, 3) versus no improvement (values 4, 5, 6, 7) and in marked improvement (values 1, 2) versus no marked improvement (values 3, 4, 5, 6, 7).

Total score of HADS-D as well as the depression and anxiety subscale scores (values and absolute change from baseline, also the change from week 16 to week 20) at all post-baseline visits.

Values of tinnitus interview (initial and follow-up) at all post-baseline visits; absolute change from baseline and change from Week 16 to Week 20 for items 8, 9, 10, 19, 20, 21, 24, 25 and 26 of the follow-up interview.

### Data Analysis

All efficacy analyses were performed on the ITT population using the last-observation-carried-forward (LOCF) approach. For sensitivity purposes an analysis of the per-protocol set and of observed cases was performed additionally. All statistical tests used for testing the primary efficacy (confirmatory testing) and secondary efficacy criteria (exploratory), and all other statistical tests used for exploratory analyses were two-sided hypothesis tests performed at the 5% significance level. For all variables standard descriptive statistics were calculated.

Change from baseline (Visit 2) to Week 16 in TBF-12 total score was analyzed using a two-way ANCOVA model with treatment group and study centers as factors and baseline TBF-12 total score as covariate.

For secondary efficacy parameters, the comparison between neramexane and placebo was performed, if appropriate, by visit using a two-way ANCOVA with treatment group and study center as factors and the corresponding baseline value of the efficacy parameter as covariate.

### Discussion

This clinical study showed promising results in terms of efficacy and safety. After a 16-week double-blind treatment (Visit 6) with final daily doses of 50 or 75 mg neramexane mesylate, patients reported a clear improvement of their tinnitus, as measured by the TBF-12, which was distinct from the groups treated with placebo or low-dose (25 mg) neramexane mesylate.

Patients with final daily doses of 50 or 75 mg after 16-weeks of double-blind treatment also reported a clear improvement of their sleep as indicated in the question 12/5 of the structured tinnitus interview, which improvement was distinct from the groups treated with placebo or low-dose (25 mg) neramexane mesylate. These results are shown in Tables 6a-6c below.

Tables 6a-6c—Analysis of question 12/5 of the structured tinnitus interview

TABLE 6a
Analysis with ITT-OC

| | Baseline | | | |
|---|---|---|---|---|
| Measure | | 25 mg/d | 50 mg/d | 75 mg/d |
| TI Sleep item | Placebo | Neramexane | Neramexane | Neramexane |
| 12/5 | N = 111 | N = 106 | N = 106 | N = 99 |
| Prevented n | 8 (7.2) | 13 (12.3) | 7 (6.6) | 8 (8.1) |
| (%) | | | | |
| affected n (%) | 82 (73.9) | 68 (64.2) | 70 (66.0) | 68 (68.7) |
| no effect n | 21 (18.9) | 25 (23.6) | 29 (27.4) | 23 (23.2) |
| (%) | | | | |

TABLE 6b
Analysis with ITT-OC

| | Week 16 Visit (Change from Baseline) | | | |
|---|---|---|---|---|
| Measure | | 25 mg/d | 50 mg/d | 75 mg/d |
| TI Sleep item | Placebo | Neramexane | Neramexane | Neramexane |
| 12/5 | N = 86 | N = 89 | N = 80 | N = 64 |
| Prevented n | 2 (2.3) | 2 (2.2) | 1 (1.3) | 4 (6.3) |
| (%) | | | | |
| affected n (%) | 57 (66.3) | 58 (65.2) | 39 (48.8) | 32 (50.0) |
| no effect n | 27 (31.4) | 29 (32.6) | 40 (50.0) | 28 (43.8) |
| (%) | | | | |

TABLE 6c
Analysis with ITT-OC

| | Follow-Up (Change from Baseline) | | | |
|---|---|---|---|---|
| Measure | | 25 mg/d | 50 mg/d | 75 mg/d |
| TI Sleep item | Placebo | Neramexane | Neramexane | Neramexane |
| 12/5 | N = 102 | N = 100 | N = 95 | N = 89 |
| Prevented n | 5 (4.9) | 3 (3.0) | 1 (1.1) | 3 (3.4) |
| (%) | | | | |
| affected n (%) | 66 (64.7) | 65 (65.0) | 55 (57.9) | 51 (57.3) |
| no effect n | 31 (30.4) | 32 (32.0) | 39 (41.1) | 35 (39.3) |
| (%) | | | | |

These findings demonstrate that, in addition to reducing tinnitus severity, neramexane has the capability to reduce sleep disturbances in patients suffering from tinnitus. Thus, neramexane may be useful in treating or preventing sleep disorders and/or in treating or preventing exacerbation of existing sleep disorders in patients suffering from tinnitus. Such sleep disorders include disorders of initiating and maintaining sleep (insomnias), disorders of the sleep-wake cycle, dysfunctions associated with sleep, sleep stages, or partial arousals (parasomnias), disorders of excessive somnolence, and non-restorative sleep.

### Example 2

### Double Blind Placebo Controlled Trial of Neramexane for Treatment of Tinnitus and Related Sleep Disorders

The objective of this project is to conduct a clinical trial to further assess the sustained effects of neramexane as a treatment for tinnitus and related sleep disorders. The primary objective of this study is to compare the efficacy, tolerability and safety of neramexane with placebo in subjects with first onset, persistent, unilateral or bilateral subjective tinnitus.

### Study Design

In a double-blind, multicenter, randomized, placebo-controlled, parallel-group study, the efficacy of neramexane in subjects suffering from tinnitus is assessed. Patients who fulfill particular inclusion criteria and meet none of particular exclusion criteria are randomized into double-blind treatment groups.

Subjects are treated for 17 weeks with neramexane or placebo including a four-resp. five-week up-titration period, depending on study drug dose, followed by a 12-week treatment-free observational period to investigate the sustained effects of the drug after cessation of the treatment.

Subjects with a target daily dose of 50 mg neramexane mesylate (<90 kg body weight) will reach steady state after four weeks, patients with a target total daily dose of 75 mg neramexane mesylate (≧90 kg body weight) will reach steady state after five weeks of treatment. For patients experiencing dose limiting adverse events with the 75 mg dose, the dosage may be reduced by switching the patient to 50 mg/day. Patients unable to tolerate a minimum dosage of 50 mg/day will be discontinued.

The scheduled visits for evaluation of each patient are as follows:

Visit 1 (screening): After signing the consent form, the subject undergoes a physical examination and clinical laboratory testing. Patient eligibility for the study is evaluated via a check of inclusion/exclusion criteria.

Visit 2 (baseline): The subject is asked about adverse events and changes in concomitant medication/disease, which events/changes are documented. The subject is evaluated for study eligibility based on a review of the inclusion/exclusion criteria. Trial procedures as well as allowed and forbidden concomitant medications are reviewed with the subject. Safety and efficacy parameters are evaluated. The subject is enrolled in the study and study medication (placebo or neramexane) is dispensed as described below.

Visit 3 (Week 5): This visit occurs at the end of the up-titration sequence. The subject is asked about adverse events and changes in concomitant medication/disease, which events/changes are documented. Safety and efficacy parameters are evaluated. Medication is dispensed as described below.

Visit 4 (Week 9): This visit occurs at the end of the first 4-week fixed-dose double-blind treatment period. The subject is asked about adverse events and changes in concomitant medication/disease, which changes are documented. Safety and efficacy parameters are evaluated. Medication is dispensed as described below.

Visit 5 (Week 13): This visit occurs at the end of the second 4-week fixed-dose double-blind treatment period. The subject is asked about adverse events and changes in concomitant medication/disease, which changes are documented. Safety and efficacy parameters are evaluated. Medication is dispensed as described below.

Visit 6 (Week 17, end of treatment). This visit occurs at the end of the 12-week fixed-dose double-blind treatment period. The subject is asked about adverse events and changes in concomitant medication/disease, which changes are documented. A clinical laboratory evaluation is performed. Safety and efficacy parameters are evaluated.

Visit 7 (Week 21): This visit occurs 4 weeks after the last study medication dose. Review of concomitant medications as well as the occurrence of adverse events since the last visit is conducted with subject. Safety and efficacy parameters are evaluated.

Visit 8 (Week 25): This visit occurs 8 weeks after the last study medication dose. Review of concomitant medications as well as the occurrence of adverse events since the last visit is conducted with subject. Safety and efficacy parameters are evaluated.

Visit 9 (Week 29): This visit occurs at the end of the 12-week follow up period after the last study medication dose. Review of concomitant medications as well as the occurrence of adverse events since the last visit is conducted with subject. Safety and efficacy parameters are evaluated.

### Administration of Neramexane

Neramexane mesylate immediate release tablets (12.5 mg and 25 mg) and matching placebo tablets are administered as film coated tablets.

Medication is dispensed from Visit 2 to Visit 5. Study medication for each study day consists of 4 separate tablets. The dosing schedule is shown in Table 7.

Throughout the double-blind treatment period, patients are to continue to take 2×2 tablets of medication daily at a constant interval of 12 hours.

TABLE 7
Administration of Neramexane mesylate

| | | | | | | 12-week fixed-dose | | 12-week |
|---|---|---|---|---|---|---|---|---|
| | 5-week double-blind up-titration period | | | | | double-blind period | | follow-up |
| Treatment group | Week 1 | Week 2 | Week 3 | Week 4 | Week 5 | Weeks 6-17 | | Weeks 18-29 |
| 75 mg/d dose | 0/12.5 | 12.5/12.5 | 12.5/25 | 25/25 | 37.5/37.5 | 37.5/37.5 | (75 mg/d) | — |
| 50 mg/d dose | 0/12.5 | 12.5/12.5 | 12.5/25 | 25/25 | 25/25 | 25/25 | (50 mg/d) | — |

| | | | | | | | |
|---|---|---|---|---|---|---|---|
| Placebo | 0/0 | 0/0 | 0/0 | 0/0 | 0/0 | 0/0 | — |

xx/xx refers to the morning/evening dose in mg, respectively

In case of dose limiting adverse events, the investigator could consider a dose reduction of 25 mg/d only in the 75 mg/d group. Subjects unable to tolerate a minimum dosage of 50 mg/d are discontinued.

Subjects are instructed to take study medication always at an individually convenient, but stable time point throughout the study course.

### Efficacy

Primary Outcome
The change in TBF-12 total score from baseline (Visit 2) at all post-baseline visits is the primary efficacy endpoint in this study.

Secondary Outcomes
TBF-12 factorial scores (values and absolute change from baseline) at all post-baseline visits.
Tinnitus loudness (11-point Likert scale).
Tinnitus annoyance (11-point Likert scale).
Tinnitus impact on life (11-point Likert scale).
Sum score of Tinnitus loudness, Tinnitus annoyance and Tinnitus impact on life (T-Score).
Sleep Questionaire (SF-B) Abridged Version scores at all post-baseline visits.

### Data Analysis

All efficacy analyses are performed on the ITT population using the last-observation-carried-forward (LOCF) approach. All statistical tests used for testing the primary efficacy (confirmatory testing) and secondary efficacy criteria (exploratory), and all statistical tests used for exploratory analyses are two-sided hypothesis tests performed at the 5% significance level.

### Discussion

This clinical study is expected to further demonstrate that neramexane has the capability for a sustained improvement of tinnitus and related sleep disorders.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference.

1-16. (canceled)

17. A method of treating or preventing sleep disorders associated with tinnitus and/or neurological diseases in a subject in need thereof, comprising administering an effective amount of a 1-aminocyclohexane derivative.

18. The method according to claim 17, wherein the 1-aminocyclohexane derivative is neramexane or a pharmaceutically acceptable salt thereof.

19. The method according to claim 18, wherein the 1-aminocyclohexane derivative is neramexane mesylate.

20. The method according to claim 19, wherein neramexane mesylate is administered in a range from about 5 mg to about 150 mg/day.

21. The method according to claim 19, wherein neramexane mesylate is administered in a range from about 5 mg to about 100 mg/day.

22. The method according to claim 19, wherein neramexane mesylate is administered at about 5 mg to about 75 mg/day.

23. The method according to claim 19, wherein neramexane mesylate is administered at about 50 mg/day.

24. The method according to claim 19, wherein neramexane mesylate is administered at about 75 mg/day.

25. The method according to claim 18, wherein neramexane or a pharmaceutically acceptable salt thereof is administered once a day, twice a day (b.i.d.), or three times a day.

26. The method according to claim 25, wherein neramexane or a pharmaceutically acceptable salt thereof is administered twice a day.

27. The method according to claim 18, wherein neramexane or a pharmaceutically acceptable salt thereof is administered in an immediate release formulation.

28. The method according to claim 18, wherein neramexane or a pharmaceutically acceptable salt thereof is administered in a modified release formulation.

29. The method according to claim 17, further comprising administration of an additional pharmaceutical agent which has been shown to be effective in treating or preventing sleep disorders.

30. The method according to claim 29, wherein the 1-amino-alkylcyclohexane derivative is neramexane or a pharmaceutically acceptable salt thereof.

31. The method according to claim 30, wherein neramexane, or a pharmaceutically acceptable salt thereof, and the additional pharmaceutical agent are administered conjointly.

32. The method according to claim 31, wherein neramexane, or a pharmaceutically acceptable salt thereof, and the additional pharmaceutical agent are administered in a single formulation.

33. The method according to claim 17, wherein said derivative is administered in a titration scheme which provides quick and safe attainment of an effective dose.

34. A pharmaceutical composition comprising a therapeutically effective amount of a 1-amino-alkylcyclohexane derivative, or a pharmaceutically acceptable salt thereof, in combination with an additional pharmaceutical agent which has been shown to be effective for the treatment or the prevention of sleep disorders and, optionally, at least one pharmaceutically acceptable carrier or excipient.

35. The pharmaceutical composition according to claim 34, wherein the 1-amino-alkylcyclohexane derivative is neramexane or a pharmaceutically acceptable salt thereof.

36. The pharmaceutical composition according to claim 34, wherein the additional pharmaceutical agent is selected from melatonin and melatonin receptor agonists.
